(19) United States
(12) Patent Application Publication
(10) Pub. No.: US 2003/0166606 A1
(43) Pub. Date: Sep. 4, 2003

(54) 5'-deoxy-n-(substituted oxycarbonyl)-5-fluorocytosine and derivatives thereof, method of preparing same, and anticancer composition comprising same as active ingredients

(76) Inventors: Kwan-Hee Kim, Kyungki-do (KR); Youn-Chul Kim, Kyungki-do (KR); Ji-Young Kim, Seoul (KR); Kyeong-Ho Lee, Kyungki-do (KR); Moon-Jong Noh, Kyungki-do (KR); Young-Seok Park, Kyungki-do (KR); Sung-Min Cho, Kyungki-do (KR); Ho-Jin Park, Sungnam-city (KR)

Correspondence Address: BIRCH STEWART KOLASCH & BIRCH, PO BOX 747, FALLS CHURCH, VA, 22040-0747, US

(21) Appl. No.: 10/333,984

(22) PCT Filed: Aug. 8, 2001
(86) PCT No.: PCT/KR01/01351

(51) Int. Cl. A61K 31/7072; C07H 19/06; C07H 19/067; C07H 19/073; C07H 19/09

(52) U.S. Cl. 514/49; 536/28.51

(57) ABSTRACT

Disclosed is a new fluorocystosine and derivatives thereof. The fluorocystosine and derivatives thereof provide a pharmaceutical composition exhibiting better anti-cancer characteristics than the conventional composition.

### BACKGROUND OF THE INVENTION

(a) Field of the Invention

The present invention relates to 5′-deoxy-N-(substituted oxycarbonyl)-5-fluorocytosine, derivatives thereof, a method of preparing the same, and an anticancer composition comprising the same as active ingredients. More particularly, the present invention relates to 5′-deoxy-N-(substituted oxycarbonyl)-5-fluorocytosine and derivatives thereof, exhibiting good anticancer activity, a method of preparing the same and an anticancer composition comprising the same as active ingredients.

(b) Related Arts

Cancer is one of the incurable diseases that are a problem to be solved in modern medical science, together with acquired immune deficiency syndrome (AIDS). The different types and number of cancer cases are increasing year by year domestically and its low cure rate causes the demise of many people. Even though a great deal of cancer research has been undertaken, suitable drugs have not yet been developed. Thus, the development of an anticancer drug with good efficacy and without side effects to more efficiently and effectively cure cancer is required.

Currently, local treatment such as a surgical operation or radiotherapy, or systematic treatment such as with chemicals or immunity treatments are used in attempts to cure cancer. Among these treatments, chemotherapy is widely used to augment local treatment procedures, or for treating cancer tumors that occur in various internal organs such as the stomach, liver, or lungs as primary cancers, as well as for various blood cancers or for reducing cancer metastasis.

Studies on anticancer drugs used in chemotherapy are actively attempting to develop various anticancer agents such as 5-fluorouracil (5-FU), methotrexate, frutraful, and cisplatin, and studies on novel anticancer drugs have also recently been undertaken. However, anticancer drugs that completely and stably cure cancer have not been developed.

Recently, investigation of an anticancer drug using 5-FU, which is one candidate of the pyrimidine neucleoside anticancer drugs, has been undertaken. 5-FU is a material to cure cancer by preventing synthesis of pyrimidine and neucleotide, but it is toxic to the stomach and intestine, and it has severe side effects. Thus, research on 5-FU derivatives with reduced side effects has been active, but new 5-FU derivatives still have a side effect causing diarrhea by activating the 5-FU derivatives in an intestinal wall after oral administration.

Recently, N⁴-alkyloxycarbonyl-5-fluorocytosine derivatives of Formula 1, which activate by enzymes in the lung rather than in the intestine and can reduce side effects, have been developed (European Patent No. 6,025,454, Japanese Patent Laid-open No. 94-211891 and U.S. Pat. No. 5,472,949).

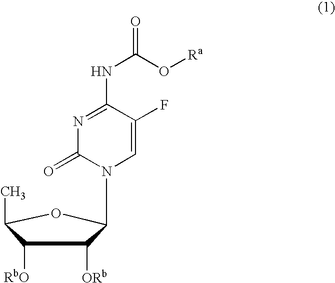

wherein, R^{a} is saturated or unsaturated hydrogen carbonate; and R^{b} is hydrogen, an easily hydrolysable radical or a protecting group easily removable under physiological conditions.

However, the derivatives have shortcomings in that anticancer activity is somewhat low. Therefore, development of new anticancer drugs exhibiting good anticancer activity is required.

### SUMMARY OF THE INVENTION

It is object to provide 5′-deoxy-N-substituted oxycarbonyl-5-fluorocytosine and derivatives thereof, exhibiting good anticancer activity.

It is another to provide a method of preparing 5′-deoxy-N-substituted oxycarbonyl-5-fluorocytosine and derivatives thereof.

It is still another to provide an anticancer composition including 5′-deoxy-N-substituted oxycarbonyl-5-fluorocytocine, or derivatives thereof, as an active ingredient.

These and other objects may be achieved by 5′-deoxy-N-substituted-oxycarbonyl-5-fluorocytosine and derivatives thereof, of Formula 2 or 3, pharmaceutically acceptable salts, or solvating materials.

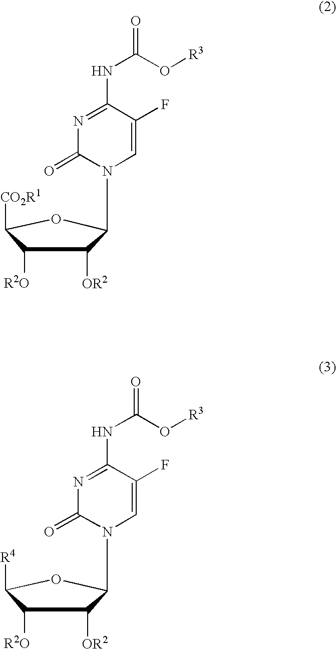

wherein, R² is an easily hydrolysable radical or a protecting group easily removable under physiological conditions; R³ is a C₁-C₇ alkyl group, alkenyl group or alkynyl group; and R⁴ is a hydroxymethyl group or a hydroxymethyl group with a protecting group.

### DETAILED DESCRIPTION OF THE INVENTION

The present invention relates to a novel compound that has good anticancer activity and is usable for an anticancer drug, and provides 5′-deoxy-N-(substituted oxycarbonyl)-5-fluorocytosine and derivatives thereof, having Formula 2 or 3 and derivatives thereof, and pharmaceutically acceptable salts or solvating materials.

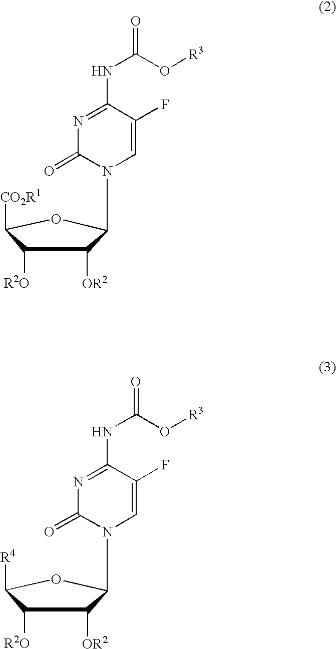

wherein, R² is an easily hydrolysable radical or a protecting group easily removable under physiological conditions, preferably hydrogen or an acetyl group; R³ is a C₁-C₇ alkyl group, alkenyl group or alkynyl group; and R₄ is a hydroxymethyl group or a hydroxymethyl group with a protecting group.

The preparation of the 5-fluorocytosine compound of Formula 2 or 3 of the present invention will be illustrated in more detail with reference to the following Reaction Formula 1.

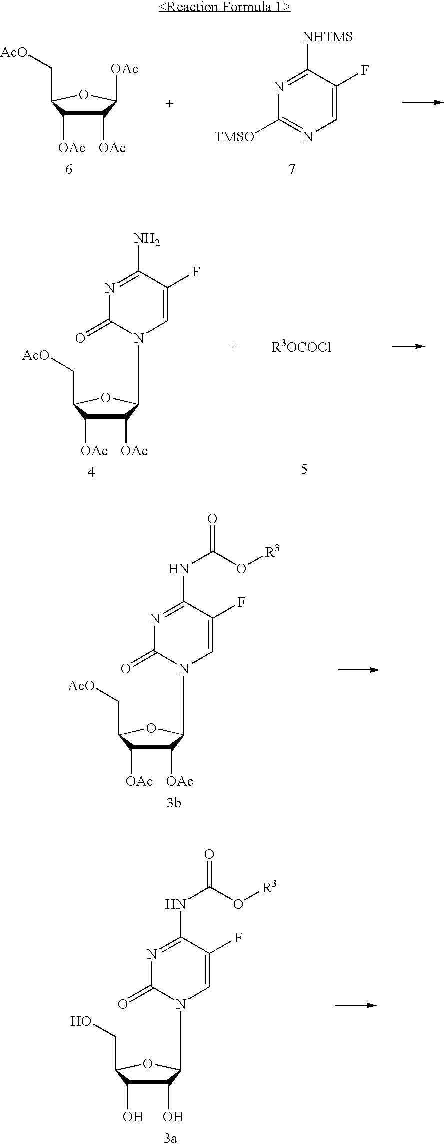

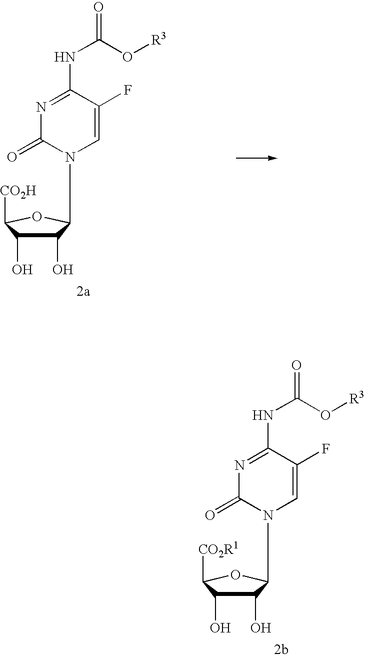

wherein, R¹ is hydrogen, a C₁-C₇ alkyl group or a C₁-C₇ alkenyl group; and R³ is a C₁-C₇ alkyl group, alkenyl group or alkynyl group.

β-D-ribofuranose 1,2,3,5-tetraacetate of Formula 6 and trimethylsilylated 5-fluorocytosine of Formula 7 are mixed in the presence of a solvent such as acetonitrile with the addition of suitable additives, e.g. titanium (IV) chloride, iodotrimethylsilane or chlorotrimethylsilane/sodium iodide, to prepare a compound of Formula 4.

Thereafter, the compound of Formula 4 (Korean application No. 2000-46179) is mixed with a compound of Formula 5 in the presence of a solvent such as methylene chloride or pyridine with the addition of a suitable base, e.g. pyridine, triethylamine, or diisopropylethylamine, to prepare a compound of Formula 3b.

The compound of Formula 3b is reduced in the presence of an alcohol such as methanol or ethanol using a C₁-C₂ sodium alkoxide or sodium hydroxide, to obtain a compound of Formula 3a.

The compound of Formula 3a reacts with oxygen gas using a suitable catalyst at room temperature to 120° C. to obtain an objective compound of Formula 2a of the present invention.

Alternatively, the compound of Formula 2a is shaken while a C₁-C₇ alkyl alcohol or alkenyl alcohol and thionyl chloride is dropped in at −30° C. to room temperature to obtain ester derivative compound of Formula 2b.

The present invention relates to an anticancer composition including 5′-deoxy-N-(substituted oxycarbonyl)-5-fluorocytosine derivatives as an active ingredient. The composition of the present invention may be variously orally or non-orally administered to patients, and the composition may include a compound of Formula 2 or 3, pharmaceutically acceptable salts, or solvating materials. The anticancer composition of the present invention may also include a physiologically acceptable liquid or solid carrier.

The solid preparation form may include powder, tablets, dispensable particles, or capsule; and a suitable solid medication type for oral administration may be a tablet, powder, or a capsule. A suitable vehicle may be a diluent, a flavor agent, a solubilizer, a lubricant, a suspension agent, a binder and/or a purification-swelling agent. If a powder or capsule preparation form is used, the carrier may include granule active components of 5 to 70%, preferably 10 to 70%. A suitable solid carrier or vehicle may be corn starch, magnesium stearate, a film, polyethylene glycol, talc, sugar, lactose, pectin, dextrin, starch, gelatin, hydroxypropylmethyl cellulose, methyl cellulose, sodium carboxymethyl cellulose, dioxide titanium, wax with low melting point, cocoa, or butter.

The liquid preparation may be a solution, a suspension, or an emulsion. For example, a non-oral injection solution includes water or mixed water-propylene glycol, and the injection has suitable isotonic properties and pH for the body system. The liquid preparation may also be polyethylene glycol aqueous solution. The suitable aqueous solution for oral administration may be prepared by dissolving active ingredients in water, and adding a flavor agent, a coloring agent, a stabilizer and a concentration-aid to the resulting material. The suitable aqueous suspension agent for oral administration may be prepared by adding particle active ingredients to a viscosity material such as natural or synthetic gum, resin, methyl cellulose, sodium carboxymethyl cellulose or conventional suspension agent.

A preferable pharmaceutical preparation is a unit administration type. The preparation includes separating a suitable amount of active ingredient into units for administration, wherein each unit of administration may be packaged as a separated amount of the preparation, for example, in a vial or ample, a tablet, a capsule, or as powder.

The present invention is further explained in more detail with reference to the following examples, but the examples are not intended to limit the present invention.

### EXAMPLE 1

### Preparation of 2′,3′,5′-tri-O-acetyl-N⁴-propargyloxycarbonyl-5-fluorocytocine (R³=propargyl, Compound of Formula 3b)

150 ml of methylene chloride and 9 ml of pyridine were added to 18.1 g of 2′,3′,5′-tri-O-acetyl-5-fluorocytosine, and 8.0 g of propargyl chloroformate was added thereto at 0° C., followed by shaking at room temperature for 30 minutes. An aqueous solution of sodium hydrogencarbonate was added to the resulting material, the obtained material was extracted three times with 300 ml of methylene chloride followed by drying under anhydrous magnesium sulfate, and filtrating. Thereafter, the obtained product was evaporated under reduced pressure to be concentrated and it was purified through silica gel column chromatography, resulting in 21.9 g of a titled product (yield 88%).

¹H NMR(CDCl₃, ppm) δ11.45 (br.s, 1H), 8.20 (t, 1H), 6.01 (s, 1H), 5.43 (m, 1H), 5.26 (m, 1H), 4.79 (dd, 2H), 4.42 (m, 1H), 4.36 (m, 2H), 2.52 (t,1H), 2.10 (s, 3H), 2.01 (s, 3H)

### EXAMPLE 2

### Preparation of N⁴-(propargyloxycarbonyl)-5-fluorocytosine (R³=propargyl, Compound of Formula 3a)

19.1 g of 2′,3′,5′-tri-O-acetyl-N⁴-propazyloxycarbonayl-5-fluorocytosine was added to and dissolved in 150 ml of methanol. 150 ml of 1N sodium methoxide was added to the resulting material, it was shaken for 1 hour, and neutralized with 1N hydrochloric acid followed by evaporation under reduced pressure to concentrate it. 100 ml of water was added to the resultant, it was extracted several times with a solution of methylene chloride/methanol (95:5), dried under anhydrous magnesium sulfate, and filtered and evaporated under reduced pressure to concentrate it. Thereafter, the concentrated material was re-crystallized using ethyl acetate to obtain 12.3 g of a titled product (yield: 80%).

¹H NMR(CD₃OD, ppm) δ8.15 (d, 1H), 5.93 (d, 1H), 4.77 (d, 2H), 4.11 (m, 1H), 3.95 (m, 1H), 3.72 (m, 3H), 2.50 (t, 1H)

### EXAMPLE 3

### Preparation of 5′-deoxy-N⁴-(propargyloxycarbonyl)-5-fluorocytosine-5′-carboxylic acid (R³=propargyl, Compound of Formula 2a)

600 ml of sodium hydrogencarbonate buffer solution with pH 8-10 was added to 10.25 g of N⁴-propargyloxycarbonyl-5-fluorocytosine, platinum oxide catalyst was added thereto, oxygen gas was injected into the resulting material at 90° C. for 12 hours, it was filtered to remove the catalyst, extracted several times with a solution of methylene chloride/methanol (95:5), dried under anhydrous magnesium sulfate, evaporated under reduced pressure to concentrate it, and it was re-crystallized using ethyl acetate to obtain 6.50 g of a titled product (yield 66%).

¹H NMR(CD₃OD, ppm) δ10.43 (br.s, 1H), 8.01 (br.s, 1H), 5.75 (d, 1H), 4.95 (d, 1H), 4.80 (d, 2H), 4.63 (m, 1H), 4.28 (m, 1H), 2.55 (t, 1H)

### EXAMPLE 4

### Preparation of ethyl-5′-deoxy-N⁴-(propargyloxycarbonyl)-5-fluorocytosine-5′-carboxylate (R¹=ethyl, R³=propargyl, Compound of 2b)

100 ml of ethanol was added to 1.0 g of 5′-deoxy-N⁴-(propargyloxycarbonyl)-5-fluorocytosine-5′-carboxylic acid, 0.6 ml of hionyl chloride was added dropwise thereto at a temperature between −30° C. to 0° C., it was shaken at room temperature for 6 to 12 hours, filtered, washed with ethanol and evaporated under reduced pressure to concentrate it. An aqueous solution of sodium hydrogencarbonate was added to the resulting material to precipitate a solid, the solid was washed with water and ethanol, and dried to obtain 1.21 g of a titled product (yield 88%).

¹H NMR(CDCl₃, ppm) δ10.35 (br.s, 1H), 8.01 (br.s, 1H), 5.77 (d, 1H), 4.88 (d, 1H), 4.81 (d, 1H), 4.29 (m, 2H), 4.12 (q, 2H), 2.50 (t, 1H), 1.24 (t,3H)

### EXAMPLE 5

### Evaluation of in vitro Anticancer Activity of 5′-deoxy-N⁴-(propargyloxycarbonyl)-5-fluorocytosine-5′-carboxylic acid (Formula 2a) Derivatives

For measuring in vitro anticancer activity of 5′-deoxy-N⁴-propargyloxycarbonyl-5-fluorocytosine-5′-carboxylic acid (R³=propargyl, compound of 2a) using 5-Fu as a reference compound, cell toxicity to human cancer cell was evaluated. The cancer cells used were A549 (lung cancer), HCT15 (colon cancer), SK-OV-3 (ovarian cancer), and SK-MEL-2 (melanoma cancer).

The cancer cells were incubated in an incubator with a constant humidity at 37° C. and 5% CO₂, and an RPMI medium with the addition of 10% fetal bovine serum was used for a basic medium. In order to identify cell toxicity, the cancer cells in a logarithmic phase were inoculated at 2-5×10⁴ cells per well of a 96-well plate, and incubated for 24 hours. A sample solution of 5-FU and 5′-deoxy-N⁴-propargyloxycarbonyl-5-flurocytosine-5′-carboxylic acid was stepwisely added to the well and incubated for 72 hours. 20 μl of 5 mg/ml MTT reacting solution dissolved in a physiological saline solution was added to the well of the incubated plate and incubated for 4 hours. The produced formazan crystal was dissolved in dimethylsulfoxide and absorbance of each well was measured at 540 nm to calculate a number of living cells. The absorbance of the well with the medium and without cell was set to 0%, that of the well without the sample was set to 100%, and the concentration corresponding to 50% was called an IC₅₀ (μg/ml) value. The results are presented in Table 1.

TABLE 1

| Concentration | | | | |
|---|---|---|---|---|
| (μg/ml) | A549 | SK-OV-3 | HCT15 | SK-MEL2 |
| 5-FU | 0.26 | 0.03 | 0.11 | 0.63 |
| Compound of | 0.005 | 0.005 | 0.040 | 0.007 |
| Example 3 | | | | |

As shown in Table 1, the compound of Formula 3 exhibits anticancer activity of about 0.04-0.007 μg/ml to each cancer cell, which is excellent compared to the control.

### EXAMPLE 6

### Evaluation of in vitro Anticancer Activity of 5′-deoxy-N⁴-(propargyloxycarbonyl)-5-fluorocytosine-5′-carboxylic acid (Formula 2a) Derivatives

Capecitabine (5′-deoxy-N⁴-(pentyloxycarbonyl)-5-fluorocytosine (in Formula 1, R^{a}=pentyl, R^{b}=hydrogen) which has good anticancer activity among 5′-deoxy-N-alkyloxycarbonyl-5-fluorocytosine derivatives disclosed in European Patent No. 602454 was used for a reference compound, 5′-deoxy-N⁴-propargyloxycarbonyl-5-fluorocytosine-5′-carboxylic acid (Formula 2a) obtained from Example 3 was used for a sample, and anticancer activity on mouse tumor cell line L1210 was measured.

The mice used were 5-week-old BDF1 male mice (19 to 20 g) that were purchased from Charles River Japan, and adapted for 1 week. The breeding condition of the mice was set to a temperature of 24±2° C. and a humidity of 50±1° C. Purified water for drinking was supplied to the mice twice a week, and straws and cages were changed once a week. The experiment was performed using two mice per group. As the tumor cell line, an L1210 mouse blood tumor cell line was used, and the cell line was sub-cultured in a Falcon culture flask twice or three times, washed with a phosphorous buffer solution (pH 7.2), and quantified under a microscope to produce a cell suspension of 1×10⁷/ml, and 100 (1×10⁶) ml of the cell suspension was abdominally administered to the mouse using 1 ml of a disposable sterilized injector. The drug was orally administered 24 hours after the cellular transplantation. The concentration of the administered capecitabine was 1.2, 5.7, 28.8, 144, and 720 mg/kg/100 μl for respective mice, and that of the compound of Formula 2a of the present invention was 1.2, 5.8, and 28.8 mg/kg/100 μl for respective mice. The capecitabine was dissolved in dimethylsulfoxide and suspended in 0.5% carboxymethyl cellulose, and the compound of Formula 2a was dissolved in distilled water. The samples were administered to the mice five times a week for 3 weeks, for a total of 15 times, starting 24 hours after the tumor cell administration. The anticancer activity was determined as increased life span to control. The results are presented in Table 2.

TABLE 2

| | | Average | |
|---|---|---|---|
| | Dosage | survival date | Increased |
| Compound | (mg/kg/day) | (day) | life span (%) |
| Control | — | 17.8 | — |
| Capecitabine | 720 | 22.8 | 30 |
| | 144 | 20.1 | 15 |
| | 28.8 | 20.2 | 14 |
| | 5.8 | 19.2 | 8 |
| Compound of | 28.8 | 21.9 | 23 |
| Example 3 | 5.8 | 20.6 | 11 |
| | 1.2 | 20.1 | 8 |

As shown in Table 2, the compound of Example 3 exhibits good anticancer activity in vivo, through the animal experiment.

As described above, the N⁴-alkynyloxycarbonyl-5-fluorocytosine and derivatives can be used for an anticancer drug.

### EXAMPLE 7

### Preparation of 2′,3′,5′-tri-O-acetyl-5-fluorocytosine 4

60 ml of acetonitrile and trimethylsilylated 5-fluorocytosine 7 obtained from 3.2 g of 5-fluorocytosine were added to 6.22 g of β-D-ribofuranose 1,2,3,5-trtraacetate 6 in the presence of nitrogen gas. 3 ml of tin (IV) chloride was added thereto at −50 to 0° C., it was shaken at room temperature for 4 to 8 hours, an aqueous solution of sodium hydrogencarbonate was added to the resulting material, it was extracted three times with 100 ml of ethyl acetate, dried under anhydrous magnesium, filtered and evaporated under reduced pressure to concentrate it, and the resultant was purified through silica gel column chromatography to obtain 6.13 g of a titled compound of Formula 4 (yield 81%).

¹H NMR(CDCl₃, ppm) δ7.65(d, 1H), 6.09(d, 1H), 5.33(dd, 1H), 5.26(dd, 1H), 4.35(m, 3H), 2.14(s, 3H), 2.08(s, 3H), 2.06(s, 3H)

### EXAMPLE 8

### Preparation of 2′,3′,5′-tri-O-acetyl-5-fluoro-N⁴-(propyloxycarbonyl)-cytosine (R³=propyl, Compound of Formula 3b)

20 ml of methylene chloride and 1.1 ml of pyridine were added to 2.33 g of 2′,3′,5′-tri-O-acetyl-5-fluorocytosine 4, 1.01 g of propyl chloroformate (R³=propyl, compound of Formula 5) was added dropwise at 0° C., it was shaken at room temperature for 30 minutes, added to an aqueous solution of sodium hydrogencarbonate, extracted three times with 50 ml of methylene chloride, dried under anhydrous magnesium sulfate, evaporated under the reduced pressure, and purified through silica gel chromatography to obtain 2.52 g of a titled compound of Formula 3b (yield 90%).

¹H NMR(CDCl₃, ppm) δ12.01(br.s, 1H), 7.58(br.s, 1H), 6.09(br.s, 1H), 5.33(d, 1H), 4.44(m, 3H), 4.12(t, 2H), 2.14(s, 3H), 2.12(s, 3H), 2.11(s, 3H), 1.61(m, 2H), 1.00(t, 3H)

### EXAMPLES 9 to 15

A compound was prepared by the same procedure in Example 8 except that a compound of Formula 5 with R³ of ethyl, butyl, pentyl, hexyl, heptyl, allyl, and propargyl was used, respectively, instead of propyl chloroformate.

### EXAMPLE 9

### 2′,3′,5′-tri-O-acetyl-5-fluoro-N⁴-(ethyloxycarbonyl)-cytoscine (R³=ethyl, Compound of Formula 3b)

¹H NMR (CDCl₃, ppm) δ12.00 (br.s, 1H), 7.55 (br.s, 1H), 6.10 (d, 1H), 5.34 (m, 2H), 4.38 (m, 3H), 4.15 (q, 2H), 2.11 (s, 3H), 2.10 (s, 3H), 2.08 (s,3H), 1.32 (t, 3H)

### EXAMPLE 10

### 2′,3′,5′-tri-O-acetyl-5-fluoro-N⁴-(butyloxycarbonyl)-cytosine (R³=butyl, Compound of Formula 3b)

¹H NMR (CDCl₃, ppm) δ12.04 (br.s, 1H), 7.54 (br.s, 1H), 6.11 (br.s, 1H), 5.30 (m, 2H), 4.40 (m, 3H), 4.14 (t, 2H), 2.13 (s, 3H), 2.11 (s, 3H), 1.69-1.25 (m, 4H), 0.90 (t, 3H)

### EXAMPLE 11

### 2′,3′,5′-tri-O-acetyl-5-fluoro-N⁴-(pentyloxycarbonyl)-cytosine R³=pentyl, Compound of Formula 3b)

¹H NMR (CDCl₃, ppm) δ12.01 (br.s, 1H), 7.61 (br.s, 1H), 6.06 (br.s, 1H), 5.27 (m, 2H), 4.37 (m, 3H), 4.12 (t, 2H), 2.15 (s, 3H), 2.10 (s, 3H), 2.08 (s, 3H), 1.71-1.31 (m, 6H), 0.92 (t, 3H)

### EXAMPLE 12

### 2′,3′,5′-tri-O-acetyl-5-fluoro-N⁴-(hexyloxycarbonyl)-cytosine (R³=hexyl, Compound of Formula 3b)

¹H NMR (CDCl₃, ppm) δ11.94 (br.s, 1H), 7.53 (d, 1H), 6.09 (d, 1H), 5.28 (m, 2H), 4.36 (m, 3H), 4.13 (t, 2H), 2.14 (s, 3H), 2.10 (s, 3H), 2.09 (s, 3H), 1.73-1.25 (m, 8H), 0.92 (t, 3H)

### EXAMPLE 13

### 2′,3′,5′-tri-O-acetyl-5-fluoro-N⁴-(heptyloxycarbonyl)-cytosine (R³=heptyl, Compound of Formula 3b)

¹H NMR (CD₃OD, ppm) δ11.94 (br.s, 1H), 7.60(d, 1H), 5.27 (m, 2H), 4.37(m, 3H), 4.14 (t, 2H), 2.15 (s, 3H), 2.13(s, 3H), 2.09(s, 3H), 1.73-1.25 (m, 10H), 0.92(t, 3H)

### EXAMPLE 14

### 2′,3′,5′-tri-O-acetyl-5-fluoro-N⁴-(allyloxycarbonyl)-cytosine (R³=allyl, Compound of Formula 3b)

¹H NMR (CD₃OD, ppm) δ12.00 (br.s, 1H), 7.63 (d, 1H), 6.10 (d, 1H), 5.89 (m, 2H), 5.27 (m, 2H), 4.77 (d, 2H), 4.39 (m, 3H), 2.13 (s, 3H), 2.12 (s, 3H), 2.10 (s, 3H)

### EXAMPLE 15

### 2′,3′,5′-tri-O-acetyl-5-fluoro-N⁴-(propargyloxycarbonyl)-cytosine (R³=propargyl, Compound of Formula 3b)

¹H NMR (CD₃OD, ppm) δ11.45 (br.s, 1H), 8.20 (t, 1H), 6.01 (s, 1H), 5.43 (m, 1H), 5.26 (m, 1H), 4.79 (dd, 2H), 4.42 (m, 1H), 4.36 (m, 2H), 2.52 (t, 1H), 2.10 (s, 3H), 2.01 (s, 3H)

### EXAMPLE 16

### Preparation of 5-fluoro-N⁴-(pentyloxycarbonyl)-cytosine (R³=pentyl, Compound of Formula 3a)

6.47 g of 2′,3′,5′-tri-O-acetyl-5-fluoro-N⁴-(pentyloxycarbonyl)-cytosine (R³=pentyl, compound of Formula 3b) was added to and dissolved in 50 ml of methanol and, 50 ml of 1N sodium methoxide, it was shaken for 1 hour, neutralized with 1N hydrochloric acid, and evaporated under the reduced pressure to concentrate. 30 ml of water was added thereto, extracted with a solution of methylene chloride/methanol (95/5) several times, dried under anhydrous magnesium, filtered, and evaporated under reduced pressure to concentrate, and re-crystallized using ethyl acetate to prepare 4.11 g of a titled product (yield 85%).

¹H NMR (CD₃OD, ppm) δ8.00 (d, 1H), 5.86 (br.s., 1H), 1.13 (t, 2H), 4.03 (m, 2H), 3.87 (dd, 1H), 3.74 (dd, 1H), 3.55 (m, 1H), 1.65 (m, 2H), 1.36 (m, 4H), 0.92 (t, 3H)

### EXAMPLES 17 to 23

A compound was prepared by the same procedure in Example 16 except that a compound of Formula 3b with R³ of ethyl, propyl, butyl, hexyl, heptyl, allyl, and propargyl was used, respectively, instead of 2′,3′,5′-tri-O-acetyl-5-fluoro-N⁴-(pentyloxycarbonyl)-cytocine.

### EXAMPLE 17

### 5-fluoro-N⁴-(ethyloxycarbonyl)-cytosine (R³=ethyl, Compound of Formula 3a)

¹H NMR (CD₃OD, ppm) δ7.98 (d, 1H), 5.88 (br.s. 1H), 4.15 (q, 2H), 4.05 (m, 2H), 3.87 (dd, 1H), 3.73 (dd, 1H), 3.56 (m, 1H), 1.30 (t, 3H)

### EXAMPLE 18

### 5-fluoro-N⁴-(propyloxycarbonyl)-cytosine (R³=propyl, Compound of Formula 3a)

¹H NMR (CD₃OD, ppm) δ7.98 (br.s, 1H), 5.86 (br.s, 1H), 4.13 (t, 2H), 4.01 (m, 2H), 3.88 (m, 1H), 3.75 (dd, 1H), 3.56 (m, 1H), 1.61 (m, 2H), 0.96 (t, 3H)

### EXAMPLE 19

### 5-fluoro-N⁴-(butyloxycarbonyl)-cytosine (R³=butyl, Compound of Formula 3a)

¹H NMR (CD₃OD, ppm) δ8.01 (d, 1H), 5.81 (br.s, 1H), 4.14 (t, 2H), 3.88 (m, 2H), 3.77 (dd, 1H), 3.56 (m, 1H), 1.57-1.33 (m, 4H), 0.95 (t, 3H)

### EXAMPLE 20

### 5-fluoro-N⁴-(hexyloxycarbonyl)-cytosine (R³=hexyl, Compound of Formula 3a)

¹H NMR (CD₃OD, ppm) δ7.95 (d, 1H), 5.80 (br.s, 1H), 4.14 (t, 2H), 4.02 (m, 2H), 3.88 (dd, 1H), 3.75 (dd, 1H), 3.55 (m, 1H), 1.57-1.29 (m, 8H), 0.94 (t, 3H)

### EXAMPLE 21

### 5-fluoro-N⁴-(heptyloxycarbonyl)-cytosine (R³=heptyl, Compound of Formula 3a)

¹H NMR (CD₃OD, ppm) δ8.03 (d, 1H), 5.88 (br.s, 1H), 4.16 (t, 2H), 4.05 (m, 2H), 3.90 (dd, 1H), 3.77 (dd, 1H), 3.56 (m, 1H), 1.57-1.61 (m, 10H), 0.94 (t, 3H)

### EXAMPLE 22

### 5-fluoro-N⁴-(allyloxycarbonyl)-cytosine (R³=allyl, Compound of Formula 3a)

¹H NMR (CD₃OD, ppm) δ8.05 (d, 1H), 5.90 (ms, 2H), 5.26 (m, 2H), 4.77 (d, 2H), 4.09 (m, 2H), 3.94 (dd, 1H), 3.78 (dd, 1H), 3.58 (m, 1H)

### EXAMPLE 23

### 5-fluoro-N⁴-(propargyloxycarbonyl)-cytosine (R³=propargyl, Compound of Formula 3a)

¹H NMR (CD₃OD, ppm) δ8.15 (d, 1H), 5.93 (d, 1H), 4.77 (d, 2H), 4.11 (m, 1H), 3.95 (m, 1H), 3.72 (m, 3H), 2.50 (t, 1H)

### EXAMPLE 24

### Preparation of 5′-deoxy-N⁴-(hexyloxycarbonyl)-5-fluorocytosine-5′-carboxylic acid (R³=hexyl, Compound of Formula 2a)

1.72 g of 5-fluoro-N⁴-(hexyloxycarbonyl)-cytosine (R³=hexyl, compound of Formula 3a) was added to 10 ml of a pH 8 to 10 sodium hydrogencarbonate buffer solution, a platinum oxide catalyst was added thereto, oxygen gas was injected into the resulting material at 90° C. for 12 hours, it was filtered to remove the catalyst, extracted several times with a methylene chloride/methanol (95/5) solution, dried under anhydrous magnesium, filtered, evaporated under the reduced pressure, and re-crystallized using ethyl acetate to obtain 1.30 g of a titled product 2a (yield 71%).

¹H NMR (CD₃OD, ppm) δ8.05 (br.s, 1H), 5.87 (d, 1H), 4.55-4.02 (m, 5H), 1.60 (m, 2H), 1.34 (m, 6H), 0.89 (t, 3H)

### EXAMPLES 25 to 31

A compound was prepared by the same procedure in Example 24 except that a compound of Formula 3a with R³ of ethyl, propyl, butyl, pentyl, heptyl, allyl and propargyl was used, respectively, instead of 5-fluoro-N⁴-(hexyloxycarbonyl)-cytosine.

### EXAMPLE 25

### Preparation of 5′-deoxy-N⁴-(ethyloxycarbonyl)-5-fluorocytosine-5′-carboxylic acid (R³=ethyl, Compound of Formula 2a)

¹H NMR (CD₃OD, ppm) δ8.08 (br.s, 1H), 5.91 (d, 1H), 4.56-4.05 (m, 5H), 1.30 (t, 3H)

### EXAMPLE 26

### Preparation of 5′-deoxy-N⁴-(propyloxycarbonyl)-5-fluorocytosine-5′-carboxylic acid (R³=propyl, Compound of Formula 2a)

¹H NMR (CD₃OD, ppm) δ8.06 (br.s, 1H), 5.89 (d,1H), 4.53-4.03 (m, 5H), 1.61 (m, 2H), 0.88 (t, 3H)

### EXAMPLE 27

### Preparation of 5′-deoxy-N⁴-(butyloxycarbonyl)-5-fluorocytosine-5′-carboxylic acid (R³=butyl, Compound of Formula 2a)

¹H NMR (CD₃OD, ppm) δ8.05 (br.s, 1H), 5.88 (d, 1H), 4.53-4.03 (m, 5H), 1.57-1.33 (m, 4H), 0.90 (t, 3H)

### EXAMPLE 28

### Preparation of 5′-deoxy-N⁴-(pentyloxycarbonyl)-5-fluorocytosine-5′-carboxylic acid (R³=pentyl, Compound of Formula 2a)

¹H NMR (CD₃OD, ppm) δ8.05 (br.s, 1H), 5.86 (d, 1H), 4.51-4.05 (m, 5H), 1.58-1.25 (m, 6H), 0.90 (t, 3H)

### EXAMPLE 29

### Preparation of 5′-deoxy-N⁴-(heptyloxycarbonyl)-5-fluorocytosine-5′-carboxylic acid (R³=heptyl, Compound of Formula 2a)

¹H NMR (CD₃OD, ppm) δ8.03 (br.s, 1H), 5.86 (d,1H), 4.53-4.03 (m, 5H), 1.60-1.28 (m, 10H), 0.91 (t, 3H)

### EXAMPLE 30

### Preparation of 5′-deoxy-N⁴-(allyloxycarbonyl)-5-fluorocytosine-5′-carboxylic acid (R³=allyl, Compound of Formula 2a)

¹H NMR (CD₃OD, ppm) δ8.11 (br.s, 1H), 5.92 (m, 2H), 5.22 (m, 2H), 1.79 (d, 2H), 4.56-4.05 (m, 3H)

### EXAMPLE 31

### Preparation of 5′-deoxy-N⁴-(propargyoxycarbonyl)-5-fluorocytosine-5′-carboxylic acid (R³=propargyl, Compound of Formula 2a)

¹H NMR (CD₃OD, ppm) δ10.43 (br.s, 1H), 8.01 (m, 2H), 5.75 (d, 1H), 4.95 (d, 1H), 4.63 (m, 1H), 4.28 (m, 1H), 2.55 (t, 1H)

### EXAMPLE 32

### Preparation of ethyl 5′-deoxy-N⁴-(hexyloxycarbonyl)-5-fluorocytosine-5′-carboxylate (R³=hexyl, Compound of Formula 2a)

1.04 g of 5′-deoxy-N⁴-(hexyloxycarbonyl)-5-fluorocytosine-5′-carboxylic acid (R³=hexyl, compound of Formula 2a) was added to 100 ml of methanol, 0.6 ml of thionyl chloride was added dropwise thereto at −30 to 0° C., it was shaken at room temperature for 6 to 12 hours, filtered, washed with ethanol, evaporated under the reduced pressure to concentrate, an aqueous solution of sodium hydrogen carbonate was added to the resulting material to precipitate solid, it was washed with water and ethanol, and dried to obtain 1.30 g of a titled product 2b (yield 92%).

¹H NMR (CDCl₃, ppm) δ8.06 (br.s, 1H), 5.90 (d, 1H), 4.55-4.02 (m, 7H), 1.64=1.09 (m, 1H), 0.91 (t, 3H)

### EXAMPLE 33

### Evaluation of in vitro Anticancer Activity of 5′-deoxy-N-alkyloxycarbonyl-5-fluorocytosine-5′-carboxylic acid (Compound of Formula 2a)

In order to measure anticancer activity of 5′-deoxy-N-alkyloxycarbonyl-5-fluorocytosine-5′-carboxylic acid (compound of Formula 2a) of the present invention using 5-FU as a control, the cell toxicity to human cancer cells was measured. The human cancer cells used were A549 (lung cancer), HCT15 (colon cancer), SK-OV-3 (ovarian cancer), and SK-MEL-2 (melanoma cancer).

The cancer cells were cultured in an incubator with a constant humidity at 37° C. and 5% CO₂, and an RPMI medium with 10% fetal bovine serum was used for a medium. In order to measure cell toxicity, the cancer cells in a logarithmic phase were inoculated into 2 to 5×10⁴ cells per well of a 96-well plate, incubated for 24 hours, and a sample solution of a stepwise dilution of 5-FU and 5′-deoxy-N⁴-pentyloxycarbonyl-5-fluorocytocine-5′-carboxylic acid were added thereto followed by incubating for 72 hours. 20 μl of 5 mg/ml of MTT reacting solution dissolved in a physiological saline solution was added to each well of the incubated plate, incubated for 4 hours, the produced formazan crystal was dissolved in dimethylsulfoxide, and the absorbance of each well was measured at a wavelength of 540 nm to calculate the number of living cells. When the absorbance of the well without the cells and with the medium was set to 0%, and that of the well without the sample was set to 100%, the concentration having the absorbance of 50% corresponding to these result was called the IC₅₀ value of the anticancer drug. These results are represented in Table 3.

TABLE 3

| | Cancer cell | | | |
|---|---|---|---|---|
| | | SK- | | |
| line | A549 | OV-3 | HCT15 | SK-MEL2 |
| 5-FU | 0.26 | 0.03 | 0.11 | 0.63 |
| Example 25 | 0.49 | 0.16 | 0.47 | 0.16 |
| (5′-deoxy-N⁴-ethyloxycarbonyl-5- | | | | |
| Example 28 | 0.17 | 0.03 | 0.12 | 0.03 |
| (5′-deoxy-N⁴-pentyloxycarbonyl-5 | | | | |
| Example 30 | 0.04 | 0.008 | 0.032 | 0.008 |
| (5′-deoxy-N⁴-allyloxycarbonyl-5- | | | | |
| Example 31 | 0.005 | 0.005 | 0.040 | 0.007 |
| (5′-deoxy-N⁴-propazylcarbonyl-5- | | | | |

As shown in Table 3, the compound of Formula 2 according to the present invention exhibits about 0.005 to 0.5 μg/ml anticancer activity to each cancer cell, which is excellent compared to the control.

### EXAMPLE 34

### Evaluation of in vivo Anticancer Activity 5′-deoxy-N⁴-(pentyloxycarbonyl)-5-fluorocytosine-5′-carboxylic acid (R³=pentyl, Compound of Formula 2a).

Capecitabine (5′-deoxy-N⁴-(pentyloxycarbonyl)-5-fluorocytosine (in Formula 1, R^{a}=pentyl, R^{b}=hydrogen) which has good anticancer activity among. 5′-deoxy-N-alkyloxycarbonyl-5-fluorocytosine derivatives disclosed in European Patent No. 602454 was used for a control, 5′-deoxy-N⁴-propazyloxycarbonyl-5-fluorocytosine-5′-carboxylic acid (Formula 2a) obtained from Example 3 was used for a sample, and anticancer activity to the mouse tumor cell line L1210 was measured.

The mice used were 5-week-old BDF1 male mice (19 to 20 g) that were purchased from Charles River Japan, and adapted for 1 week. The breeding condition of the mouse was set to a temperature of 24±2° C. and a humidity of 50±1° C. Purified water for drinking was supplied to the mice twice a week, and straws and cages were changed once a week. The experiment was performed using two mice per group. As the tumor cell line, an L1210 mouse blood tumor cell line was used, the cell line was sub-cultured in a Falcon culture flask twice or three times, washed with a phosphorous buffer solution (pH 7.2), quantified under a microscope to produce a cell suspension of 1×10⁷/ml, and 100 (1×10⁶) ml of the cell suspension was abdominally administered to the mouse using 1 ml of a disposable sterilized injector. The drug was orally administered 24 hours after the cellular transplantation. The concentration of the administered capecitabine was 1.2, 5.7, 28.8, 144, and 720 mg/kg/100 μl for respective mice, and that of the compound of 2a of the present invention was 1.2, 5.8, 28.8 mg/kg/100 μl for respective mice. The capecitabine was dissolved in dimethylsulfoxide and suspended in 0.5% carboxymethyl cellulose, and the compound of 2a was dissolved in distilled water. The samples were administered to the mice five times a week for 3 weeks, for a total of 15 times, starting 24 hours after the tumor cell administration. The anticancer activity was determined as increased life span to control. The results are presented in Table 4.

TABLE 4
Evaluation of anticancer activity in vivo

| | | Average life | |
|---|---|---|---|
| | Dosage | maintenance | Increased |
| Compound | (mmol/kg/day) | day (days) | life span (%) |
| Control | — | 8.8 | — |
| Capecitabine | 1.5 | 12.5 | 42.0 |
| | 0.67 | 9.6 | 9.1 |
| | 0.13 | 9.4 | 6.8 |
| Example 28 | 1.5 | 16.8 | 91.9 |
| (5′-deoxy-N⁴-(pentyloxyc- | 0.67 | 11.3 | 28.4 |
| arbonyl)-5-fluorocytosine- | 0.13 | 9.1 | 3.4 |
| 5′-carboxylic acid) | | | |
| 5-FU | 0.23 | 16.5 | 87.5 |
| | 0.15 | 14.3 | 62.5 |

### EXAMPLE 35

### Evaluation of in vivo Anticancer Activity of 5′-deoxy-N-alkyloxycarbonayl-5-fluorocytosine-5′-carboxylic acid (Compound of Formula 2a) Derivatives

Capecitabine (5′-deoxy-N⁴-(pentyloxycarbonyl)-5-fluorocytosine (in Formula 1, R^{a}=pentyl, R^{b}=hydrogen) which has good anticancer activity among 5′-deoxy-N-alkyloxycarbonyl-5-fluorocytosine derivatives disclosed in European Patent No. 602454 was used for a reference compound, 5′-deoxy-N⁴-propazyloxycarbonyl-5-fluorocytosine-5′-carboxylic acid (Formula 2a) obtained from Example 3 was used for a sample, and anticancer activity to mouse tumor cell line L1210 was measured.

The mice used were 5-week-old BDF1 male mice (19 to 20 g) that were purchased from Charles River Japan, and adapted for 1 week. The breeding condition of the mice was set to a temperature of 24±2° C. and a humidity of 50±1° C. Purified water for drinking water was supplied to the mice twice a week, and straws and cages were changed once a week. The experiment was performed using two mice per group. As the tumor cell line, an L1210 mouse blood tumor cell line was used, the cell line was sub-cultured in a Falcon culture flask twice or three times, washed with a phosphorous buffer solution (pH 7.2), quantified under a microscope to produce a cell suspension of 1×10⁷/ml, and 100 (1×10⁶) ml of the cell suspension was abdominally administered to the mouse using 1 ml of a disposable sterilized injector. The drug was orally administered 24 hours after the cellular transplantation. The concentration of the injected capecitabine was 1.2, 5.7, 28.8, 144, and 720 mg/kg/100 μl for respective mice, and that of the compound of Formula 2a of the present invention was 1.2, 5.8, 28.8 mg/kg/100 μl for respective mice. The capecitabine was dissolved in dimethylsulfoxide and suspended in 0.5% carboxymethyl cellulose, and the compound of 2a was dissolved in distilled water. The samples were administered to the mice five times a week for 3 weeks, for a total of 15 times, starting 24 hours after the tumor cell administration. The anticancer activity was determined as increased life span. The results are presented in Table 5.

TABLE 5
Evaluation of anticancer activity in vivo

| | | Average | Life |
|---|---|---|---|
| | Dosage | life day | maintenance |
| Compound | (mg/kg/day) | (days) | rate (%) |
| Control | — | 17.8 | — |
| Capecitabine | 720 | 22.8 | 30 |
| | 144 | 20.1 | 15 |
| | 28.8 | 20.2 | 14 |
| | 5.8 | 19.2 | 8 |
| | 1.2 | 18.9 | 6 |
| Example 30 | 28.8 | 25.7 | 44 |
| (5′-deoxy-N⁴-(allyloxycarbonyl)- | 5.8 | 24.8 | 39 |
| 5-fluorocytosine-5′-carboxylic | 1.2 | 22.1 | 25 |
| acid) | | | |
| Example 31 | 28.8 | 21.9 | 23 |
| (5′-deoxy-N⁴- | 5.8 | 20.6 | 11 |
| (propazyloxycarbonyl)- | | | |
| 5-fluorocytosine-5′-carboxylic | 1.2 | 20.1 | 8 |
| acid) | | | |

As shown in Tables 4 and 5, the compound of Formula 2 according to the present invention exhibits excellent anticancer activity through animal tests.

What is claimed is:

1. 5-fluorocytosine and derivatives thereof, having Formula 2, pharmaceutically acceptable salts, or solvating materials: 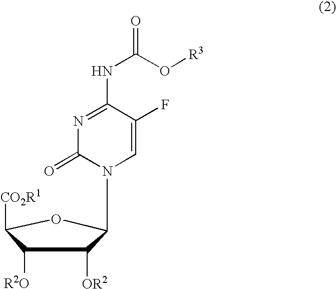
wherein, R¹ is hydrogen, a C₁-C₇ alkyl group or a C₁-C₇ alkynyl group; R² is an easily hydrolysable radical or a protecting group easily removable under physiological conditions; and R³ is a C₂-C₇ alkyl group, alkenyl group or alkynyl group.

2. 5-fluorocytosine and derivatives thereof, having formula 3, pharmaceutically acceptable salts, or solvating materials: 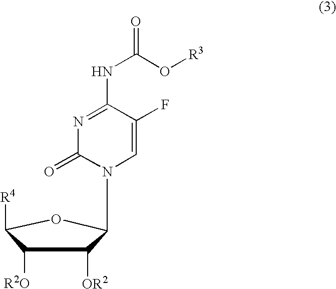
wherein, R² is an easily hydrolysable radical or a protecting group easily removable under physiological conditions; R³ is a C₁-C₇ alkyl group, alkyenyl group, or alkynyl group; and R⁴ is a hydroxymethyl group or a hydroxymethyl group with a protecting group.

3. A method of preparing 5-fluorocytosine and derivatives thereof, having Formula 2a, comprising:
reacting a compound of Formula 3a with a base to prepare a compound of Formula 3b;
oxidizing the compound of Formula 3a. 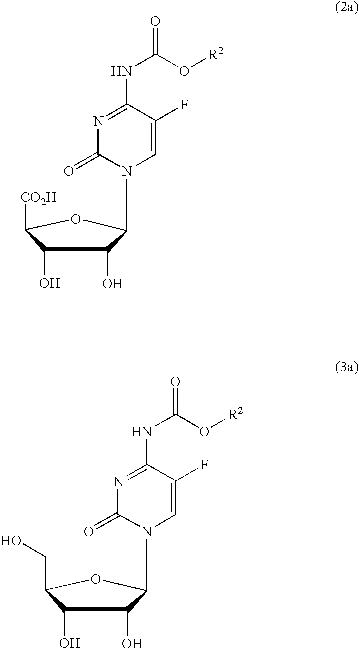
wherein, R² is an easily hydrolysable radical or a protecting group easily removable under physiological conditions; R³ is a C₁-C₇ alkyl group, alkyenyl group, or alkynyl group; and R⁴ is a hydroxymethyl group or a hydroxymethyl group with a protecting group.

4. A method of preparing 5-fluorocytosine or derivatives thereof, having Formula 2b, comprising esterifying a compound of Formula 2a. 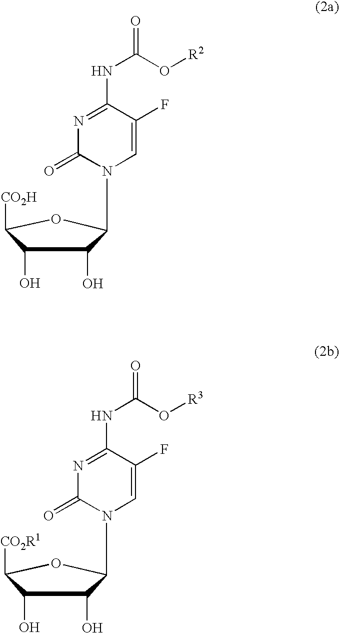
wherein, R² is an easily hydrolysable radical or a protecting group easily removable under physiological conditions; R³ is a C₁-C₇ alkyl group, alkenyl group or alkynyl group; and R⁴ is a hydroxymethyl group or a hydroxymethyl group with a protecting group.

5. A method of preparing 5-fluorocytosine and derivatives thereof, having Formula 3b, comprising reacting a compound of Formula 4 with a compound of Formula 5 in the presence of a base. 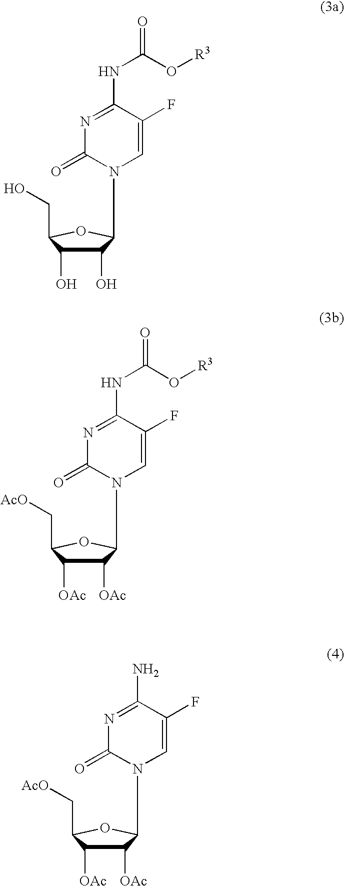
wherein, R² is an easily hydrolysable radical or a protecting group easily removable under physiological conditions; R³ is a C₁-C₇ alkyl group, alkenyl group or alkynyl group; R⁴ is a hydroxymethyl group or a hydroxymethyl group with a protecting group.

6. The method of claim 5 wherein the base is pyridine, triethylamine or diisopropylethylamine.

7. An anticancer composition comprising 5-fluorocytosine and derivatives thereof, having Formula 2 or 3. 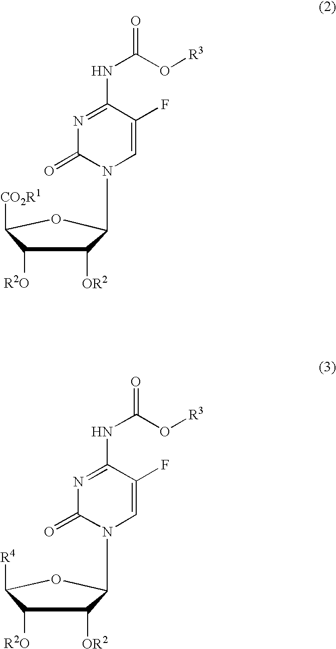
wherein, R² is an easily hydrolysable radical or a protecting group easily removable under physiological conditions; preferably a hydrogen or an acetyl group; R³ is a C₁-C₇ alkyl group, alkenyl group, or alkynyl group; and R⁴ is a hydroxymethyl group or a hydroxymethyl group with a protecting group.
